(19) United States
(12) Patent Application Publication
(10) Pub. No.: US 2003/0105162 A1
(43) Pub. Date: Jun. 5, 2003

(54) Method for treating bronchial constriction and bronchospasm

(75) Inventor: Alain Martin, Ringoes, NJ (US)

Correspondence Address: Richard R. Muccino, 758 Springfield Avenue, Summit, NJ, 07901, US

(73) Assignee: Celluar Sciences, Inc., Flemington, NJ

(21) Appl. No.: 10/205,354

(22) Filed: Jul. 25, 2002

Related U.S. Application Data
(60) Provisional application No. 60/313,871, filed on Aug. 21, 2001.

(51) Int. Cl. A61K 31/28; A61K 31/19; A61K 31/195

(52) U.S. Cl. 514/557; 514/561; 514/492; 514/495

(57) ABSTRACT

The present invention is directed to a method for treating bronchial constriction in mammals. The method comprises contacting mammalian lung with a compound selected from the group consisting of alpha-keto acids having four or more carbon atoms and precursors of alpha-keto acids having four or more carbon atoms. The compound is present in a therapeutically effective amount to produce bronchial dilation. The present invention is also directed to a method for treating bronchial constriction in mammals. The method comprises contacting mammalian lung with a compound selected from the group consisting of alpha-keto acids having four or more carbon atoms and precursors of an alpha-keto acids having four or more carbon atoms. The compound is present in an amount from about 0.0001 millimoles to about 0.01 millimoles. The present invention is further directed to a method for treating bronchial spasm in mammals. The method comprises contacting mammalian lung with a compound selected from the group consisting of alpha-keto acids having four or more carbon atoms and precursors of alpha-keto acids having four or more carbon atoms. The compound is present in a therapeutically effective amount to reduce bronchial spasm. The present invention is still further directed to a method for treating airway disease in mammals. The method comprises contacting mammalian lung with a compound selected from the group consisting of alpha-keto acids having four or more carbon atoms and precursor of alpha-keto acids having four or more carbon atoms. The compound is present in a therapeutically effective amount to prevent bronchial spasm. The present invention is still further directed to a method for treating airway disease in mammals. The method comprises contacting mammalian lung with a compound selected from the group consisting of alpha-keto acids having four or more carbon atoms and precursors of alpha-keto acids having four or more carbon atoms. The compound is present in a therapeutically effective amount to prevent bronchial constriction.

This application claims priority from provisional application serial No. 60/313,871, filed Aug. 21, 2002.

### FIELD OF THE INVENTION

This invention pertains to therapeutic methods of preventing and treating bronchial spasm and bronchial constriction. This invention also pertains to compounds used in the therapeutic methods.

### BACKGROUND OF THE INVENTION

More than 17 million people in the U.S. now have asthma, an increase of more than 75 percent since 1980. As the number of patients has risen, so have the larger consequences of the disease. Today, asthma is one of the top reasons for hospitalization of children. It causes children to miss more than 10 million school days a year and adults to miss 3 million days at work. It is responsible for more than 10 million doctor visits a year. It is estimated that asthma will be responsible for more than 5,600 deaths this year, more than twice as many as 20 years ago.

Chronic Obstructive Pulmonary Disease (COPD) is a blockage of airflow out of the lungs. COPD encompasses emphysema, alpha antritrypsin deficiency-related (AAT) emphysema, and chronic bronchitis. Nearly 16 million Americans suffer from COPD, which is the fourth leading cause of death, claiming the lives of nearly 87,000 Americans annually.

Smoking causes approximately 80 to 90 percent of COPD cases; a smoker is 10 times more likely than a nonsmoker to die of COPD. Other known causes are frequent lung infections and exposure to air pollutants. Depending on the severity of the disease, treatments may include bronchial dilators which open up air passages in the lungs; antibiotics; and exercise to strengthen muscles. People with COPD may eventually require supplemental oxygen and may have to rely on mechanical respiratory assistance.

Emphysema causes irreversible lung damage. The walls between the air sacs within the lungs lose their ability to stretch and recoil. They become weakened and break. Elasticity of the lung tissue is lost, causing air to be trapped in the air sacs and impairing the exchange of oxygen and carbon dioxide. An estimated 1.9 million Americans have emphysema.

Symptoms of emphysema include cough, shortness of breath and an increased effort to breathe. Diagnosis is by pulmonary function tests, along with the patient's history, examination and other tests. The quality of life for a person suffering from emphysema diminishes as the disease progresses. At the onset, there is minimal shortness of breath. Eventually, there is severe shortness of breath often leading to the total dependency on the administration of oxygen around the clock.

Alpha antitrypsin deficiency-related (AAT) emphysema, also called “early onset emphysema,” is caused by the inherited deficiency of a protein called alpha 1-antitrypsin (AAT) or alpha-protease inhibitor. AAT, produce by the liver, is a “lung protector.” In the absence of AAT, emphysema is inevitable. An estimated 50,000 to 100,000 American have AAT deficiency emphysema, primarily of northern European descent.

The onset of AAT deficiency emphysema is characterized by shortness of breath, decreased exercise capacity. Blood screening is used if the trait is known to be in the family and will determine if a person is a carrier or AAT-deficient. If children are diagnoses as AAT-deficient through blood screening, they may undergo a liver transplant to prevent the onset of AAT deficiency emphysema in their adult life.

Chronic bronchitis is an inflammation of the lining of the bronchial tubes. An estimated 13.8 million people suffer from chronic bronchitis, the sixth leading chronic condition in America. Whereas emphysema is more concentrated in the elderly, chronic bronchitis affects people of all ages. Symptoms include chronic cough, increased mucus, frequent clearing of the throat and shortness of breath. It may precede or accompany pulmonary emphysema. Treatments aimed at reducing irritation in the bronchial tubes include antibiotics and bronchial dilators.

Airway diseases such as asthma, acute bronchitis, emphysema, chronic obstructive emphysema, centrilobular emphysema, panacinar emphysema, chronic obstructive bronchitis, reactive airway disease, cystic fibrosis, bronchiectasis, acquired bronchiectasis, kartaagener's syndrome, atelectasis, acute atelectasis, chronic atelectasis, pneumonia, legionnaires disease, psittacosis, fibrogenic dust disease, diseases due to organic dust, diseases due to irritant gases and chemicals, hypersensitivity diseases of the lung, idiopathic infiltrative diseases of the lungs and the like are generally characterized by cough, shortness of breath and an increased effort to breath.

U.S. Pat. Nos. 5,798,388; 5,939,459 and 5,952,384 issued to Katz. The Katz inventions pertain to a method for treating various disease states in mammals caused by mammalian cells involved in the inflammatory response and compositions useful in the method. The method comprises: contacting the mammalian cells participating in the inflammatory response with an inflammatory mediator; wherein the inflammatory mediator is present in an amount capable of reducing the undesired inflammatory response and is an antioxidant. The preferred inflammatory mediator is a pyruvate. Katz discloses the treatment of airway diseases of the lungs such as bronchial asthma, acute bronchitis, emphysema, chronic obstructive emphysema, centrilobular emphysema, panacinar emphysema, chronic obstructive bronchitis, reactive airway disease, cystic fibrosis, bronchiectasis, acquired bronchiectasis, kartaagener's syndrome, atelectasis, acute atelectasis, chronic atelectasis, pneumonia, essential thrombocytopenia, legionnaires disease, psittacosis, fibrogenic dust disease, diseases due to organic dust, diseases due to irritant gases and chemicals, hypersensitivity diseases of the lung, idiopathic infiltrative diseases of the lungs and the like by inhaling pyruvate containing compositions. The pyruvate acts as an inflammatory response mediator and reduces the undesired inflammatory response in mammalian cells.

U.S. Pat. No. 5,296,370 (Martin et al.) discloses therapeutic compositions for preventing and reducing injury to mammalian cells and increasing the resuscitation rate of injured mammalian cells. In one embodiment, the therapeutic composition comprises (a) pyruvate selected from the group consisting of pyruvic acid, pharmaceutically acceptable salts of pyruvic acid, and mixtures thereof, (b) an antioxidant, and (c) a mixture of saturated and unsaturated fatty acids wherein the fatty acids are those fatty acids required for the resuscitation of injured mammalian cells.

U.S. Pat. No. 5,547,946 (Molinari) discloses topical pharmaceutical compositions in the form of oral, local and/or nasal liquid solution or suspension, for instillation, inhalation or insufflation. The pharmaceutical compositions contain as an active agent a compound selected from the group consisting of D, L-alpha-glycerophosphoric acid, glutaric acid and their sodium or potassium salts as the essential active agent for use in the treatment of respiratory allergies, allergic rhinitis, allergic conjunctivitis, allergic asthma or allergy to fur or dust and in which free ion calcium plays a role. The active agent, when supplied in a sufficient amount, is effective to enable by a reduction in free ion calcium concentration the removal or improvement in symptoms of allergy amenable to said free calcium ion concentration reduction.

While the above therapeutic compositions and methods are reported to treat various conditions, none of the compositions and methods disclose or teach the bronchial dilating or bronchial spasm preventing properties of low doses of pyruvate or pyruvate precursors nor do they disclose methods of treating airway diseases with a bronchial dilating effective amount or bronchial spasm preventing amount of a pyruvate or pyruvate precursor.

### SUMMARY OF THE INVENTION

The present invention pertains to a method for treating an airway condition in mammals which may be characterized by one or more of the following most common symptoms: chronic cough, increased mucus, frequent clearing of the throat, wheezing, chest tightness, coughing, gasping for breath, shortness of breath and other conditions related to bronchial constriction and bronchial spasm. The method for treating such an airway condition in mammals comprises contacting the lungs with a compound selected from the group consisting of an alpha-keto acid having four or more carbon atoms and a precursor of an alpha-keto acid having four or more carbon atoms; wherein the compound is present in an amount capable of producing bronchial dilation. The invention further comprises a method for treating airway disease in mammals which comprises contacting the mammalian lung with a compound selected from the group consisting of an alpha-keto acid having four or more carbon atoms and a precursor of an alpha-keto acid having four or more carbon atoms; wherein the compound is present in a therapeutically effective amount to prevent bronchial spasm.

### DETAILED DESCRIPTION OF THE INVENTION

Conditions having the symptoms of chronic cough, increased mucus, frequent clearing of the throat, wheezing, chest tightness, coughing, gasping for breath, shortness of breath and other conditions related to bronchial constriction and bronchial spasm are often caused by over reaction to a stimuli. Such a stimuli can be classed as a trigger or an inducer and vary from person to person. The triggers irritate the airways and result in bronchial constriction and bronchial spasm. Common triggers include but are not limited to: cold air, dust, strong fumes, exercise, inhaled irritants, emotional upsets and smoke. Inducers may also cause bronchial constriction. Typical inducers include allergens and respiratory viral infections. Allergens include but are not limited to: pollen, animal secretions, molds and house dust mites. Exposure to inducers not only results in bronchial constriction, but can also lead to inflammation and serious lung disorders.

During bronchial constriction, the muscles in the bronchial tubes constrict, causing difficulty in breathing. Airflow through these passages becomes difficult resulting in labored breathing. This is often followed by increased mucous secretions, which further plug the airways. Bronchial constriction and increased mucus may result in cough and wheezing. With less and less air available through the lungs, oxygen in the blood decreases.

It has been found that an alpha-keto acid having four or more carbon atoms and a precursor of an alpha-keto acid having four or more carbon atoms act as a bronchial dilator in mammals with bronchial constriction. It is believed that when an alpha-keto acid having four or more carbon atoms or a precursor of an alpha-keto acid having four or more carbon atoms is applied to lung tissue, hereinafter the bronchial dilator, the bronchial dilator acts outside of the lung cells. Without being held to a specific theory of operation, it is believed that the extra cellular bronchial dilators act as a reactive oxygen species antagonist reducing the active oxygen species present in the lung. It is believed these reactive oxygen species are either directly or indirectly responsible for bronchial constriction and bronchial spasm. When the active oxygen agents are removed, the lungs return to normal. In reducing the active oxygen species present in the lung, the Bronchial dilator is consumed. Bronchial spasm is a series of short duration bronchial constrictions alternating with periods of bronchial relaxation. Additionally, the alpha-keto acids may act to enhance the lung's ability to remove mucus thereby clearing the lungs allowing less obstructed airflow. The alpha-keto-acids of the present may reduce the viscosity of mucus by removing hydrogen peroxide, which is known to thicken mucus. The alpha-keto-acids may also act as surfactants further reducing the viscosity of mucus thus facilitating its removal through the normal bodily processes of expulsion and absorption. The removal of mucus may also reduce the triggers present in the lung thereby reducing and/or preventing bronchial constriction and bronchial spasm.

In a preferred embodiment, the therapeutic compositions containing the Bronchial dilator are administered locally to the bronchi. In another preferred embodiment, the therapeutic compositions are administered systemically. In yet another preferred embodiment, the therapeutic compositions are administered systemically and locally concomitantly. The bronchial dilator is administered at a concentration so as to produce no toxic response or no more that a minimal irritation in the mammal being treated.

In a preferred embodiment, the therapeutic compositions are administered by inhalation. The therapeutic compositions may be first nebulized by any suitable means. The means of delivering the medicine to the lungs may be for example by nebulizer or metered dose inhalers (MDI's). Such MDI's may use propellants such as gases or they may be dry powder inhalers or mini-nebulizers. The therapeutic compositions may be in liquid or solid form with liquid droplets or particle size being small enough to facilitate access to the bronchi by inhalation.

In another preferred embodiment, a sterile solution of Bronchial dilator is nebulized and inhaled by the patient. A therapeutically effective amount of Bronchial dilator is inhaled. This may be accomplished in a single inhalation or by repeated inhalations over a period of time of about 1 to 30 minutes. Preferably, inhalation will be complete in less than 20 minutes. Most preferably inhalation will be complete in less than 15 minutes.

The preferred Bronchial dilator is at least one compound selected from the group consisting of an alpha-keto acid having four or more carbon atoms and a precursor of an alpha-keto acid having four or more carbon atoms. A precursor is a substance from which another substance is formed and in this text also includes salts. The preferred Bronchial dilator will prevent bronchial constriction.

The preferred Bronchial dilator is at least one compound selected from the group consisting of an alpha-keto acid having four or more carbon atoms and a precursor of an alpha-keto acid having four or more carbon atoms. A precursor is a substance from which another substance is formed and in this text also includes salts. The preferred Bronchial dilator will prevent bronchial spasm.

Preferably the alpha-keto acid is selected from the group consisting of oxaloacetic acid, ketoglutaric acid, ketobutyric acid, ketoadipic acid, ketocaproic acid, ketoisovaleric acid, their salts and mixtures thereof. The salt may be selected from the group consisting of aluminum, ammonium, lithium, sodium, potassium, magnesium, calcium, zinc, manganese, and the like and mixtures thereof. Sodium, potassium and calcium are the most preferred salts.

Another preferred Bronchial dilator is selected from the group precursors consisting of alpha-keto acids of four or more carbon atoms in combinations of alpha-keto acid-glycine, alpha-keto acid-cystine, alpha-keto acid-alanine, alpha-keto acid-leucine, alpha-keto acid-valine, alpha-keto acid-isoleucine, alpha-keto acid-phenylalanine and alpha-keto amide.

Compositions for treating airway disease in mammals comprise a compound for preventing bronchial spasm and a carrier composition. The carrier composition is selected from the group consisting of tablets, capsules, powders, liquids, isotonic liquids, isotonic media, microparticultes and the like.

The bronchial dilator is administered in a therapeutically effective amount to reduce the undesired bronchial constriction or to prevent bronchial constriction. In the typical case, the bronchial dilator is administered from about 0.0001 to about 0.05 millimoles per dose, preferably about 0.0005 to about 0.03 millimole per dose, more preferably about 0.0005 to about 0.01 millimoles per dose, still more preferably about 0.0005 to about 0.005 millimoles per dose, still more preferably about 0.0005 to about 0.0035, and most preferably about 0.001 to about 0.003 millimoles per dose. A millimole of Bronchial dilator is the equivalent weight of one millimole of alpha-keto acid anion or approximately. A 5 ml solution of 0.5 millimeter concentration Bronchial dilator will contain 0.0025 millimoles of alpha-keto acid anion.

Typical airway diseases causing bronchial spasm, bronchial constriction or both treatable by the present compositions and method include but are not limited to acute bronchitis, asthma, emphysema, chronic obstructive emphysema, chronic obstructive pulmonary disease, centrilobular emphysema, panacinar emphysema, chronic obstructive bronchitis, reactive airway disease, cystic fibrosis, bronchiectasis, acquired bronchiectasis, interstitial lung disease, kartaagener's syndrome, atelectasis, acute atelectasis, chronic atelectasis, pneumonia, legionnaires disease, psittacosis, fibrogenic dust disease, diseases due to organic dust, diseases due to irritant gases and chemicals, hypersensitivity diseases of the lung, idiopathic infiltrative diseases of the lungs, chronic obstructive pulmonary disease and the like.

The bronchial dilator of the present invention may be administered prior to, after and/or with other therapeutic agents. Typical therapeutic agents are antibacterials, antivirals, antifungals, antihistamines, bronchial dilators, leukotriene receptor antagonists, proteins, enzymes, hormones, nonsteroidal anti-inflammatories, cytokines, steroids, and the like.

It is understood that the method of administration and the condition being treated will greatly affect the dose required to achieve the desired therapeutic effect. A mild asthmatic would be expected to respond to a lower dose than a severe asthmatic.

### EXAMPLE

Response of Mild Asthmatic to Bronchial Dilator Treatment

The sodium salts of the following keto-acids: oxaloacetic acid, ketoglutaric acid, ketobutyric acid, ketoadipic acid, ketocaproic acid, ketoisovaleric acid, were prepared as 5 mM solutions in normal saline. Each keto-acid solution and a normal saline control were filter sterilized by passing it through a sterile 0.2 micron filter. The sterile solution to be tested was placed into a “pulmo Aid” nebulizer manufactured by DeVilbiss Co., Somerset, Pa. 15501-0635. The sterile solution was then nebulized by the Pulmo Aid device fitted with a disposable nebulizer and inhaled by the patient. The nebulizer produces a steady stream of nebulized liquid into the mouth piece of the disposable nebulizer. The patient inhaled and exhaled normally from through the mouthpiece of the nebulizer until all of the test solution was nebulized. This process typically took about ten (10) to twenty (20) minutes. The subject was tested as described below about 15 minutes after completing inhalation of each test solution.

A three ball incentive deep breather exerciser was used to evaluate the ability of the test subject to inhale before treatment and after treatment with normal saline (baseline) and after treatment with the 5 mM test solution. The incentive deep breather exerciser, Triflow II #717301, Sherwood Medical, Saint Louis, Mo., was used for all testing. This device has three balls in three parallel chambers. The balls become suspended in the incoming stream of air when a patient inhales through the mouthpiece. A non-asthmatic will suspend all three balls with each inhalation. This is equivalent to the subject inhaling about 1000 to 1200 cc of air per second. Each test consists of three inhalation readings taken about 30 seconds apart. The highest reading is taken as the result for each inhalation test.

On a normal day, the asthmatic test subject could only inhale strong enough to raise one or two balls. This is equivalent to about 600 to 900 cc of air per second. Treatment with saline did not enhance the test subject's ability to inhale. Treatment with each of the keto-acids tested did enhanced breathing of the test subject to the normal range of 1000 to 1200 cc per second.

The bronchial dilator albuterol was tested as a positive control. After inhalation of albuterol, the test subject was able to inhale strong enough to raise all three balls in the Triflow device.

On each test day, the test subject was first treated with the saline control and his ability to inhale tested 15 minutes later. Then the active test solution was administered as described above and the test subject's ability to inhale was tested again 15 minutes later.

Conclusion: Each of the keto-acids tested was as effective a bronchial dilator as albuterol.

While the method for treating the bronchial constriction or bronchial spasm herein described constitute preferred embodiments of this invention, it is to be understood that the invention is not limited to this precise form of method and that changes may be made therein without departing from the scope of the invention which is defined in the appended claims.

I claim:

1. A method for treating bronchial constriction in mammals comprising contacting mammalian lung with a compound selected from the group consisting of alpha-keto acids having four or more carbon atoms and precursors of alpha-keto acids having four or more carbon atoms; wherein the compound is present in a therapeutically effective amount to produce bronchial dilation.

2. The method of claim 1 wherein the compound is inhaled.

3. The method of claim 1 wherein the compound is present in an amount from about 0.0001 millimoles to about 0.05 millimoles.

4. The method of claim 1 wherein the compound is present in an amount from about 0.0005 millimoles to about 0.03 millimoles.

5. The method of claim 1 wherein the compound is present in an amount from about 0.0005 millimoles to about 0.01 millimoles.

6. The method of claim 1 wherein the compound is present in an amount from about 0.0005 millimoles to about 0.005 millimoles.

7. The method of claim 1 wherein the compound is present in an amount from about 0.0005 millimoles to about 0.003 millimoles.

8. The method of claim 1 wherein the compound is present in an amount from about 0.001 millimoles to about 0.0035 millimoles.

9. The method of claim 1 wherein the compound is a salt selected from the group consisting of aluminum, ammonium, lithium, sodium, potassium, magnesium, calcium, zinc, manganese, and the like and mixtures thereof of a compound selected from the group consisting of an alpha-keto acid having four or more carbon atoms and a precursor of an alpha-keto acid having four or more carbon atoms.

10. The method of claim 1 wherein the compound precursor is selected from the group consisting of alpha-keto acids of four or more carbon atoms in combinations of alpha-keto acid-glycine, alpha-keto acid-cystine, alpha-keto acid-alanine, alpha-keto acid-leucine, alpha-keto acid-valine, alpha-keto acid-isoleucine, alpha-keto acid-phenylalanine and alpha-keto amide, and salts thereof.

11. The method of claim 1 wherein the alpha-keto acid is selected from the group consisting of oxaloacetic acid, ketoglutaric acid, ketobutyric acid, ketoadipic acid, ketocaproic acid, ketoisovaleric acid, their salts and mixtures thereof

12. The method of claim 1 further comprising contacting the mammalian lung with a therapeutic agent.

13. The method of claim 12 wherein the therapeutic agent is administered prior to the compound.

14. The method of claim 12 wherein the therapeutic agent is administered concomitantly with administration of the compound.

15. The method of claim 12 wherein the therapeutic agent is administered after administration of the compound.

16. The method of claim 12 wherein the therapeutic agent is one or more agents selected from the group consisting of antibacterials, antivirals, antifungals, antihistamines, bronchial dilators, leukotriene receptor antagonists, proteins, enzymes, hormones, nonsteroidal anti-inflammatories, cytokines and steroids.

17. A method for treating bronchial constriction in mammals comprising contacting mammalian lung with a compound selected from the group consisting of alpha-keto acids having four or more carbon atoms and precursors of alpha-keto acids having four or more carbon atoms; wherein the compound is present in an amount from about 0.0001 millimoles to about 0.01 millimoles.

18. A method for treating bronchial spasm in mammals comprising contacting mammalian lung with a compound selected from the group consisting of alpha-keto acids having four or more carbon atoms and precursors of an alpha-keto acids having four or more carbon atoms; wherein the compound is present in a therapeutically effective amount to reduce bronchial spasm.

19. The method of claim 18 wherein the compound is inhaled.

20. The method of claim 18 wherein the compound is present in an amount from about 0.0001 millimoles to about 0.05 millimoles.

21. The method of claim 18 wherein the compound is present in an amount from about 0.0005 millimoles to about 0.03 millimoles.

22. The method of claim 18 wherein the compound is present in an amount from about 0.0005 millimoles to about 0.01 millimoles.

23. The method of claim 18 wherein the compound is present in an amount from about 0.0005 millimoles to about 0.005 millimoles.

24. The method of claim 18 wherein the compound is present in an amount from about 0.0005 millimoles to about 0.003 millimoles.

25. The method of claim 18 wherein the compound is present in an amount from about 0.001 millimoles to about 0.0035 millimoles.

26. A method for treating airway disease in mammals comprising contacting mammalian lung with a compound selected from the group consisting of alpha-keto acids having four or more carbon atoms and precursors of alpha-keto acids having four or more carbon atoms; wherein the compound is present in a therapeutically effective amount to prevent bronchial spasm.

27. A method for treating airway disease in mammals comprising contacting mammalian lung with a compound selected from the group consisting of alpha-keto acids having four or more carbon atoms and precursors of alpha-keto acids having four or more carbon atoms; wherein the compound is present in a therapeutically effective amount to prevent bronchial constriction.
